(19) United States
(12) Patent Application Publication
(10) Pub. No.: US 2010/0203175 A1
(43) Pub. Date: Aug. 12, 2010

(54) DEGLYCATION OF AGEs

(75) Inventors: Nabil ABDUL-MALAK, Caluire (FR); Cecile ALTOBELLI, Lyon (FR); Eric PERRIER, Les Cotes d'Arey (FR)

Correspondence Address: BASF Beauty Care Solutions France, 100 Campus Drive, Florham Park, NJ, 07932, US

(73) Assignee: BASF BEAUTY CARE SOLUTIONS FRANCE S.A.S., Lyon (FR)

(21) Appl. No.: 12/668,061

(22) PCT Filed: Jul. 9, 2008
(86) PCT No.: PCT/EP08/58955

§ 371 Date: Mar. 10, 2010

(30) Foreign Application Priority Data
Jul. 9, 2007 (FR) 0756350

(51) Int. Cl. A61K 36/67; A61K 31/192; A61K 31/352; A61K 31/404; A61K 31/05; A61K 31/135; A61K 31/60; A61K 31/403; A61K 31/35; A61K 36/00; A61K 8/97; A61K 36/736; A61K 8/36; A61K 8/49; A61K 8/34; G01N 33/68; A61Q 19/00; A61P 17/00

(52) U.S. Cl. 424/734; 514/570; 514/456; 514/418; 514/731; 514/646; 514/159; 514/411; 514/460; 424/725; 424/769; 424/735; 436/86

(57) ABSTRACT

The invention relates to the use, as an active ingredient, of at least one substance that promotes the deglycation of AGEs for preparing a composition, especially for limiting the presence of AGEs in a tissue.

The invention particularly relates to the use of such a substance for preparing a composition intended to prevent and/or combat a reduction in flexibility and/or plasticity and/or elasticity and/or functionality of a tissue, and/or to prevent and/or combat the ageing of a tissue, by promoting the deglycation of AGEs in the tissue, said tissue preferably being the skin, or the tissue wall of a blood vessel or of an organ.

The invention relates to the use of active principles for promoting the deglycation of glycated proteins, in other words to promote the reversion of the Maillard reaction.

The present invention particularly relates to substances that can be used topically or orally to act on the glycated proteins in a tissue, such as for example in the skin, or in the tissue wall of a blood vessel or of an organ, especially in a human being.

### PRIOR ART

It is known in the prior art that sugar, especially in the form of glycans induces glycation of proteins (bonding of sugar to the proteins), especially in the skin. This glycation is, however, completely normal when the blood sugar level increases, such as, in particular in diabetic subjects, during ageing, or when the food is rich in sugars.

It is known in the prior art that non-enzymatic glycosylation or glycation is a purely chemical and spontaneous reaction that consists in covalently bonding a carbohydrate to a peptide chain.

Glycation is a fundamental mechanism of ageing that results from the attachment of free sugars to amino acids or to proteins.

Glycated proteins are also known as advanced glycation end products or AGEs. These compounds, in particular, reduce the flexibility, elasticity and functionality of the skin.

The glycation process occurs in three steps:
1—Formation of a Schiff base resulting from the attachment of a reducing sugar (glucose, ribose or fructose) or of an aldehyde to the amino acid residues of the protein, mainly lysine and the N-terminal amine fraction.
2—Molecular rearrangement, known as Amadori rearrangement, resulting from isomerization of the Schiff base. The rate of formation of these Amadori products is proportional to the sugar concentration.
3—Slow and irreversible accumulation, via rearrangements, hydrogen transfers and formation of very reactive intermediates, of glycation end products or Maillard products. This reaction leads to the formation of AGEs, better known by the expression Maillard products. The rate of formation of these compounds is independent of the sugar concentration of the medium but depends on the duration of hyperglycaemia and on the rate of protein turnover.

The first two stages (Schiff base and Amadori rearrangement) stabilize at a plateau and can be reversed depending on the level of glycaemia. On the other hand, the third stage progresses regardless of the level of glycaemia.

Extracellular matrix proteins, the lifetime of which in the body is very long, are affected by glycation. Glycation modifies the properties of these proteins, making them more resistant to proteolysis and preventing their turnover. Furthermore, the AGEs induce the formation of molecular bridges between the collagen fibres, making them more rigid and less soluble. Finally, the AGEs might have other actions by bonding themselves to specific receptors present in macrophages, endothelial and mesangial cells, by inducing the secretion of proinflammatory cytokines or growth factors. The importance of the glycation of proteins has been emphasized by the effect of drugs which inhibit glycation, which is expressed by a slowing down of the ageing of certain functions in laboratory animals. In the course of diabetes mellitus an excessive glycation of the proteins also occurs, which is linked to the rise in glycaemia.

The solutions proposed in the prior art relate to active substances that make it possible to limit the glycation of proteins and also the formation of AGEs.

### OBJECTIVES OF THE INVENTION

The main objective of the invention is to solve the technical problem consisting of the provision of active substances that make it possible to limit the presence of AGEs, by reversing the Maillard reaction, especially in a tissue, such as for example in the skin, or in the tissue wall of a blood vessel or of an organ, and in particular in the skin.

One particular objective of the present invention is to solve this technical problem within the context of the provision of cosmetic, dermatological, or pharmaceutical compositions that prevent or combat the reduction of the elastic and plastic properties of a tissue, such as for example in the skin, or in the tissue wall of a blood vessel or of an organ, and in particular in the skin, especially during ageing of the tissues or during diabetes.

Another objective of the present invention is to provide a method of screening active principles that have the aforementioned properties.

One particular objective of the present invention is to solve the technical problem in a reliable and reproducible manner by providing non-toxic active substances, in particular for the cosmetics, dermatological, dermopharmaceutical or pharmaceutical industry, preferably that can be applied topically.

One particular objective of the invention is to provide substances that have a low toxicity and that are dermatologically acceptable.

Another objective of the present invention is to provide active principles, the preparation of which is inexpensive and can be carried out on an industrial scale in a reliable and simple manner.

### DESCRIPTION OF THE INVENTION

Glycation is involved in numerous progressive diseases linked to ageing, such as vascular diseases (for instance atherosclerosis), kidney disease, arthritis, the complications of diabetes, cicatrization, etc. It is significant that the diabetic complications due to glycation can occur even at a younger age in diabetic individuals, whose average blood sugar concentration is higher than normal.

In diabetes, during which the involvement of free radicals is widely documented, the oxidative stress is directly linked to hyperglycaemia. The sugars that are present in a too large amount in the blood then oxidize easily. This oxidation of the sugars leads, inter alia, to sugar/protein grafts or glycation. The increased level of glycated haemoglobin in the case of diabetes is the typical example.

The glycation products, the level of which is proportional to the level of glycaemia, over a long period of time, are partly responsible for tissue ageing, of which the reduction of the elastic and plastic properties is one of the causes.

Thus, the present invention describes the use of an active substance that promotes the deglycation of AGEs, or that promotes the reversion of the Maillard reaction with respect to AGEs, for the preparation of a composition, especially for limiting the presence of AGEs in a tissue.

Advantageously, this composition is intended, for example, to combat the reduction in flexibility and/or elasticity and/or plasticity and/or functionality of a tissue, such as for example in the skin, or in the tissue wall of a blood vessel or of an organ.

The present invention also relates to a composition for combating and/or preventing ageing of the skin, for preventing and/or combating the reduction in the elastic and plastic properties of tissues, and in particular of the skin, via deglycation of AGEs, or by reversion of the Maillard reaction with respect to AGEs, in cutaneous tissue, comprising, as an active substance, a substance that promotes the deglycation of AGEs.

The present invention relates to the use of at least one active substance that promotes the deglycation of AGEs, or that promotes the reversion of the Maillard reaction with respect to AGEs, chosen from the following group: 3,5-dimethoxy-4-hydroxycinnamic acid (sinapic acid), trans-3,3′,4′,5,7-pentahydroxyflavane (catechin), oxindole, 3,4-dihydroxyphenylacetic acid (DOPAC), 1,3,5-trihydroxybenzene (phloroglucinol), 4-aminophenol, 2-hydroxybenzoic acid (salicylic acid), 6-hydroxy-2,5,7,8-tetramethylchroman-2-carboxylic acid (trolox), nordihydroguaiaretic acid, 6-methyl-5,7a,13,13b-tetraazapentaphene-8,14-dione, 6-hydroxy-indole, salsolinol hydrobromide, 7-hydroxy-4,6-dimethyl phthalide, 3,4-dihydroxycinnamic acid (caffeic acid), physostigmine, 2-hydroxymethyl-5-hydroxy-γ-pyrone (kojic acid), and also the plant extracts containing them, at least one extract of a plant chosen from the group consisting of: Awati, piper mistletoe, blue vervain, Cecropia, wintergreen, cherry tree, Cassia and Indian shot; and any mixture thereof for preparing a composition to promote the deglycation of AGEs.

Preferably, the active substance that promotes the deglycation of AGEs, or that promotes the reversion of the Maillard reaction with respect to AGEs is a plant extract chosen from the group consisting of:
awati (Maprounea guyanensis), and preferably the leaves;
Jamaica vervain (Stachytarpheta jamaicensis);
Cecropia (Cecropia obtusa), and preferably the leaves and/or the buds; and
wintergreen (Chimaphila umbellata).

According to one alternative preferred embodiment, the active substance that promotes the deglycation of AGEs, or that promotes the reversion of the Maillard reaction with respect to AGEs is a characterized molecule chosen from the group consisting of:
3,5-dimethoxy-4-hydroxycinnamic acid (sinapic acid);
trans-3,3′,4′,5,7-pentahydroxyflavane (catechin);
oxindole;
3,4-dihydroxyphenylacetic acid (DOPAC);
1,3,5-trihydroxybenzene (phloroglucinol);
   and mixtures thereof, and also the plant extracts containing them.

Advantageously, the composition is a cosmetic, dermatological, dermopharmaceutical or pharmaceutical composition preferably that can be applied topically, or a food (nutraceutical) supplement.

The present invention relates to the use of an active substance for preparing a composition to combat the glycation of glycated proteins, especially in cutaneous tissue, linked to the rise in glycaemia during diabetes mellitus.

The present invention relates to the use of an active substance for preparing a composition to combat the AGEs formed in glomeruli, and especially to reduce the excretion of albumin in a diabetic subject.

The active substance may be concentrated by freeze drying, spray-drying, etc.

Said active substance is generally used at a concentration between 0.001 and 10%, preferably between 0.01 and 5%, and more particularly at 1% for plant extracts, and between 1×10⁻⁷ and 1%, preferably between 1×10⁻⁷ and 1×10⁻¹%, more preferably between 1×10⁻⁵ and 1×10⁻¹%, for the characterized molecules, by weight of the total composition, without this limiting the concentration to be used.

Among all of these active substances, those which promote the deglycation of AGEs at least by 50% with reference to the action produced by 15 mM aminoguanidine are preferred.

The present invention also relates to a cosmetic composition that can be applied topically or as a food supplement, or a pharmaceutical composition especially intended for preventing and/or combating the reduction in flexibility and/or plasticity and/or elasticity and/or functionality of the skin, and/or for preventing and/or combating skin ageing, by promoting the reversion of the Maillard reaction with respect to AGEs in the skin, said composition comprising, as an active ingredient, at least one substance that promotes the deglycation of AGEs in the skin, preferably chosen from the group consisting of: 3,5-dimethoxy-4-hydroxycinnamic acid (sinapic acid), trans-3,3′,4′,5,7-pentahydroxyflavane (catechin), oxindole, 3,4-dihydroxyphenylacetic acid (DOPAC), 1,3,5-trihydroxybenzene (phloroglucinol), 4-aminophenol, 2-hydroxybenzoic acid (salicylic acid), 6-hydroxy-2,5,7,8-tetramethylchroman-2-carboxylic acid (trolox), nordihydroguaiaretic acid, 6-methyl-5,7a,13,13b-tetraazapentaphene-8,14-dione, 6-hydroxyindole, salsolinol hydrobromide, 7-hydroxy-4,6-dimethyl phthalide, physostigmine, and any mixture thereof, or at least one extract of a plant chosen from the group consisting of: awati, piper mistletoe, Jamaica vervain, Cecropia, wintergreen, cherry tree, Cassia, and Indian shot; optionally as a mixture with a compound that inhibits glycation, especially by mineralization, such as EDTA derivatives, phytic acid or azelaic acid, or that promotes the protection of skin proteins, such as aminoguanidine, guanidine, or enzyme inhibitors, such as pentosidine, or substances that reduce the amount of sugar available to participate in the glycation reaction, such as carnosine, ascorbic acid or α-tocopherol.

In one preferred composition according to the present invention, the active substance that promotes the deglycation of AGEs, or that promotes the reversion of the Maillard reaction with respect to AGEs is a plant extract chosen from the group consisting of:
awati (Maprounea guyanensis), and preferably the leaves;
Jamaica vervain (Stachytarpheta jamaicensis);
Cecropia (Cecropia obtusa), and preferably the leaves and/or the buds; and
wintergreen (Chimaphila umbellata).

According to one alternative preferred embodiment, the active substance that promotes the deglycation of AGEs, or that promotes the reversion of the Maillard reaction with respect to AGEs is a characterized molecule chosen from the group consisting of:
3,5-dimethoxy-4-hydroxycinnamic acid (sinapic acid);
trans-3,3′,4′,5,7-pentahydroxyflavane (catechin);
oxindole;
3,4-dihydroxyphenylacetic acid (DOPAC);
1,3,5-trihydroxybenzene (phloroglucinol).

Depending on the plants, it is advantageous to use the whole plant or part of the plant chosen from a root, rhizome, stem, husk, flower, fruit, seed, germ, and leaf, preferably at 1 to 10% (w/w) in a solvent or a mixture of solvents, preferably a polar protic solvent, and advantageously in water, an alcohol, a glycol, a polyol, a water/alcohol, water/glycol or water/polyol mixture (such as water mixed with ethanol, glycerol, butylene glycol or other glycols, such as xylitol, etc.) of 100/0 to 0/100 (v/v). The extracts obtained are then preferably filtered or distilled in order to recover the soluble fraction which is then filtered. The active substance is advantageously the plant extract in a solvent, such as water, an alcohol, polyol, glycol, or a mixture thereof, preferably diluted to a concentration between 0.01 and 10% (v/v).

Among the compounds that inhibit glycation (that is to say that reduce the formation of AGEs), use may also be made of a compound extracted, preferably by maceration in a solvent or mixture of solvents such as water, an alcohol, a glycol, a polyol, or a mixture of these solvents, and preferably water, (use is preferably made of compounds that are soluble in these solvents, preferably after filtration), of a plant chosen from: an extract of guarana (Paullinia cupana), preferably the seeds, epimedium (Epimedium brevicornum), preferably the leaves, curled dock (Rumex crispus), preferably the husk, sarsaparilla (Smilax ornata), preferably the root, indigenous vine (Davilla rugosa), preferably the leaves, catechu (wood preferably), milk thistle (fruit preferably), pine, preferably Pinus species (root preferably), Chinese rhubarb (root preferably), hawthorn (leaf preferably), leucocyanidins (seeds preferably), areca (seeds preferably), bilberry (fruit preferably), elder (fruit preferably), walnut (leaf preferably), willow (bark preferably), lettuce (leaf preferably) and lespedeza (leaf preferably); and/or a compound chosen from sodium erythrosine, 1,4-anthraquinone, catechol, 4-hydroxychalcone, 4-aminophenol, an OPC (PCO procyanidolic oligomer), such as strawberry plant OPC, prunin, and 1-amino-2-hydroxymethylanthraquinone, or a mixtures of these substances.

It is most particularly advantageous to combine the active substances that make it possible to limit the presence of AGEs, by reversing the Maillard reaction, with at least one of the active substances above that inhibits glycation, preferably chosen from an extract of guarana (Paullinia cupana), preferably the seeds, epimedium (Epimedium brevicornum), preferably the leaves, curled dock (Rumex crispus), preferably the husk, sarsaparilla (Smilax ornata), preferably the root, indigenous vine (Davilla rugosa), preferably the leaves, 3,5-dimethoxy-4-hydroxycinnamic acid (sinapic acid), trans-3,3′,4′,5,7-pentahydroxyflavane (catechin), oxindole, 3,4-dihydroxyphenylacetic acid (DOPAC), 1,3,5-trihydroxybenzene (phloroglucinol), taken individually or in combination.

Thus, the present invention relates to a cosmetic care method comprising the topical application, or application as a food (nutraceutical) supplement, of a composition comprising, as a cosmetic active principle, at least one of the aforementioned active ingredients or active ingredients that are mentioned below.

The present invention also relates to a method of treating the human body comprising the administration of an aforementioned pharmaceutical composition or a pharmaceutical composition that is mentioned below, preferably by topical application, to prevent and/or combat the glycation of proteins in a tissue, especially when the blood sugar level rises and/or is high, such as for example during diabetes. The invention especially relates to a method of reducing the excretion of albumin in a diabetic subject.

The present invention also relates to a method of screening active principles that promote the deglycation of AGEs, comprising:
a) the production of AGEs;
b) bringing the AGEs into contact with a substance to be screened for its activity with respect to the deglycation of AGEs; and
c) the selection of at least one active principle that promotes the deglycation of AGEs.

Advantageously, step a) comprises an incubation of at least one type of protein of the skin or of the walls of blood vessels in the presence of a sugar (for example: glucose, ribose, fructose, etc.) under conditions that allow the formation of AGEs, and preferably at a temperature between 40 and 60° C., and more preferably at around 50° C., for a duration between 1 and 5 weeks and preferably 3 weeks.

Advantageously, step b) comprises the incubation of AGEs in the presence of at least one substance to be screened at a temperature between 40 and 60° C., preferably at around 50° C.

The selection of the active principle is carried out by comparison of the results obtained in the presence of the active principle tested compared to a control.

The compounds according to the present invention are prepared in the form of topical compositions, especially cosmetic, dermopharmaceutical or pharmaceutical compositions. Therefore, for these compositions, the excipient contains, for example, at least one compound chosen from the group consisting of preservatives, emollients, emulsifiers, surfactants, moisturizers, thickeners, conditioners, mattifying agents, stabilizers, antioxidants, texturizing agents, brighteners, film-forming agents, solubilizers, pigments, dyes, fragrances and sunscreens.

These excipients are preferably chosen from the group consisting of amino acids and derivatives thereof, polyglycerols, esters, polymers and derivatives of cellulose, lanolin derivatives, phospholipids, lactoferrins, lactoperoxidases, sucrose-based stabilizers, the E vitamin and derivatives thereof, natural and synthetic waxes, vegetal oils, triglycerides, unsaponifiable matter, phytosterols, plant esters, silicones and derivatives thereof, protein hydrolysates, jojoba oil and derivatives thereof, fatsoluble/water-soluble esters, betaines, aminoxides, plant extracts, saccharose esters, titanium dioxides, glycines, and parabens, and more preferably from the group consisting of butylene glycol, steareth-2, steareth-21, glycol-15 stearyl ether, cetearyl alcohol, phenoxyethanol, methylparaben, ethylparaben, propylparaben, butylparaben, butylene glycol, natural tocopherols, glycerin, sodium dihydroxycetyl phosphate, isopropyl hydroxycetyl ether, glycol stearate, triiso-nonanoin, octyl cocoate, polyacrylamide, isoparaffin, laureth-7, a carbomer, propylene glycol, glycerol, bisabolol, a dimethicone, sodium hydroxide, PEG-30 dipolyhydroxystearate, capric/caprylic triglycerides, cetearyl octanoate, dibutyladipate, grapeseed oil, jojoba oil, magnesium sulphate, EDTA, a cyclomethicone, xanthan gum, citric acid, sodium laurylsulphate, mineral oils and waxes, isostearyl isostearate, propylene glycol dipelargonate, propylene glycol isostearate, PEG-8 beeswax, glycerides of hydrogenated palm kernel oil, glycerides of hydrogenated palm oil, lanolin oil, sesame oil, cetyl lactate, lanolin alcohol, castor oil, titanium dioxide, lactose, saccharose, low-density polyethylene and an isotonic saline solution.

It is particularly advantageous to combine the active substances according to the present invention with other active agents having complementary properties to further improve the efficacy against skin ageing, against the reduction in flexibility and/or plasticity and/or elasticity and/or functionality of the skin. Advantageously, these other active agents are chosen from:
the active substances stimulating cell proliferation and/or differentiation that have an anti-ageing effect, especially the following molecules: NGF, alpha-MSH, beta-endorphin or derivatives thereof, especially those described in Patent Application FR 2857874;
the active substances protecting the fibroblast growth factor (FGF) especially FGF2, in particular those described in the Patent Application in the name of the Applicant published under the No. GB 244036, in particular an extract of Hibiscus abelmoschus;
the active substances stimulating the activity and/or proliferation of fibroblasts, especially a fermented soy peptide, in particular that sold by the Applicant under the name Phytokine™, advantageously in combination with an extract of Hibiscus abelmoschus;
the active substances stimulating hyaluronase synthase, especially HAS2, in particular those described in Patent FR 2893252;
the active substances stimulating the activity and/or the synthesis of lysyl oxidase, especially LOX, in particular those described in Patent Application FR 2855968, and preferably the dill extract; and
the active substances having antiinflammatory properties, such as those that inhibit PLA2, in particular an extract of Pueraria lobata roots such as described in Patent FR 2847267 (Inhipase®).

Advantageously, the aforementioned compositions are formulated in a form chosen from the group consisting of an aqueous or oily solution, a cream or an aqueous gel or an oily gel, especially in a pot or in a tube, especially a shower gel or a shampoo; a milk; an emulsion, a microemulsion or a nanoemulsion, especially of the oil-in-water or water-in-oil or multiple or silicone-based type; a lotion, especially in a glass or plastic bottle or in a measuring bottle or in an aerosol; an ampoule; a liquid soap; a dermatological bar; an ointment; a foam; an anhydrous product that is preferably liquid, pasty or solid, for example in stick form, especially in the form of a lipstick.

The expression “topical application” used here means to apply or spray the composition according to the present invention over the surface of the skin.

The expression “dermatologically acceptable” used here means that the composition or the components of the latter are suitable for use in contact with human skin without causing toxicity, incompatibility, instability, allergic response or their equivalents.

The expressions “that promote the deglycation of AGEs” or “that favour the reversion of the Maillard reaction with respect to AGEs” mean that the substance makes it possible to obtain, from an advanced glycation end product (AGE), a non-glycated protein in an amount greater than a negative control (absence of active principle), for example as assessed by a difference in fluorescence according to Example 1 between a control sample (absence of active principle) and a sample containing the substance that promotes the deglycation of AGEs. Preferably, the fluorescence obtained with the sample containing the substance that promotes the deglycation of AGEs is compared to the fluorescence obtained assessed in the presence of aminoguanidine. The expression “substance that promotes the deglycation of AGEs” in particular relates to substances for which the fluorescence obtained in the presence of the substance is at least equal to 70% of the fluorescence obtained in the presence of 15 mM aminoguanidine and in the absence of the active principle. The expression “deglycation of AGEs” means the reversion of the Maillard reaction.

Many cosmetically active ingredients are known by a person skilled in the art for improving the health and/or the physical appearance of the skin. A person skilled in the art knows how to formulate the cosmetic or dermatological compositions to obtain the best effects. On the other hand, the compounds described in the present invention may have a synergic effect when they are combined with one another. These combinations are also covered by the present invention. The CTFA Cosmetic Ingredient Handbook, Second Edition (1992) describes various cosmetic and pharmaceutical ingredients commonly used in the cosmetic and pharmaceutical industries which are suitable in particular for topical use. Examples of these classes of ingredients include, without being limited thereto, the following compounds: abrasives, absorbents, compounds having an aesthetic purpose such as fragrances, pigments, dyes, essential oils, astringents, etc. (for example: clove oil, menthol, camphor, eucalyptus oil, eugenol, menthyl lactate, witch hazel distillate), anti-acne agents, anti-flocculating agents, antifoaming agents, antimicrobial agents (for example: iodopropyl butylcarbamate), antioxidants, binders, biological additives, buffering agents, swelling agents, chelating agents, additives, biocidal agents, denaturants, external analgesics, film-forming materials, polymers, opacifiers, pH adjusters, reducing agents, depigmenting or lightening agents (for example: hydroquinone, kojic acid, ascorbic acid, magnesium ascorbylphosphate, ascorbyl-glucosamine), conditioning agents (for example: humectants), skin-soothing agents and/or wound-healing agents (for example: panthenol and derivatives thereof (for example: ethylpanthenol), aloe vera, pantothenic acid and derivatives thereof, allantoin, bisabolol, and dipotassium glycyrrhizinate), thickeners, and vitamins, and derivatives or equivalents of the latter.

Other objectives, features and advantages of the invention will appear clearly to a person skilled in the art after reading the explanatory description which refers to examples which are given solely by way of illustration and which should in no way limit the scope of the invention.

The examples are an integral part of the present invention and any feature that appears novel with respect to any prior art based on the description taken in its entirety, including the examples, is an integral part of the invention both functionally and generally.

Thus, each example has a general scope.

Moreover, in the examples, all the percentages are given by weight, unless indicated otherwise, and the temperature is expressed in degrees Celsius unless indicated otherwise, and the pressure is atmospheric pressure, unless indicated otherwise.

### EXAMPLES

### Example 1

### Reversion of the Maillard Reaction

1—Preparation of AGEs:

The preparation of a solution containing a protein and a reducing sugar was carried out extemporaneously as follows: a solution of bovine serum albumin (BSA) at a concentration between 1.5 μM and 1.5M, preferably between 15 μM and 500 μM was incubated with a solution of a reducing sugar such as glucose, fructose, etc., preferably with glucose at a concentration between 0.1M and 10M and preferably between 0.5M and 5M.

The solution of bovine serum albumin may be substituted by human collagen.

The incubation was maintained between 1 and 5 weeks, and preferably for 3 weeks.

The incubation temperature was maintained between 20 and 70° C., preferably at 50° C.
2—Incubation of AGEs with the potentially active compound and detection of the reversion phenomenon of the Maillard reaction:

The solution containing the AGEs prepared in 1 was incubated with or without (negative control) the presence of a compound to be tested for its deglycation activity of AGEs, at a temperature between 40 and 60° C., preferably at around 50° C. for 1 to 5 weeks, preferably for 3 weeks. The compounds were tested at a concentration between 0.001 and 10%, preferably between 0.01 and 5%, and more particularly at 1% by weight of the total composition. The positive control used was aminoguanidine at a concentration between 15 μM and 150 mM, preferably at a concentration between 1.5 and 15 mM. The measure of inhibition of the AGEs was carried out by measuring the fluorescence (excitation wavelength between 350 and 375 nm, preferably at 355 nm; emission wavelength between 420 and 450 nm, preferably at 430 nm). The reversion of the Maillard reaction was calculated by comparing the fluorescence of AGEs set at 100% (maximum fluorescence) with the fluorescence of AGEs+active agent tested.

### Example 2

### Deglycation of AGEs by Characterized Molecules

The preparation of a protein/reducing sugar solution was carried out according to the method described in Example 1 (paragraph 1). The molecules were tested at a concentration between 1×10⁻⁷ and 1×10⁻¹%, preferably between 1×10⁻⁵ and 1×10¹%, and in particular at 1×10⁻¹%, for example in water or DMSO, as described in Example 1 (paragraph 2).

The molecules that promote the deglycation of AGEs under the conditions described, in a manner at least equivalent to that of aminoguanidine used at 15 mM were:

TABLE 1

| | | | % |
|---|---|---|---|
| | | | degly- |
| Common name | INCI | CAS | cation |
| 3,5-Dimethoxy-4-hydroxycinnamic | | 530-59-6 | 100 |
| acid (sinapic acid) | | | |
| Oxindole | | 59-48-3 | 100 |
| 4-Aminophenol | p-Amino- | 123-30-8 | 100 |
| | phenol | | |
| trans-3,3′,4′,5,7- | | 7295-85-4 | 100 |
| Pentahydroxyflavane (catechin) | | | |
| 3,4-Dihydroxyphenylacetic acid | | 102-32-9 | 100 |
| (DOPAC) | | | |
| 1,3,5-Trihydroxybenzene | | 108-73-6 | 100 |
| (phloroglucinol) | | | |
| 2-Hydroxybenzoic acid | | 69-72-7 | 100 |
| (salicylic acid) | | | |
| 6-Hydroxy-2,5,7,8-tetramethyl- | | | 100 |
| chromane-2-carboxylic acid | | | |
| (trolox) | | | |
| Nordihydroguaiaretic acid | | 500-38-9 | 100 |
| 6-Methyl-5,7a,13,13b- | | | 100 |
| tetraazapentaphene-8,14-dione | | | |
| Indole alcohol amine II | 6-Hydroxy- | | 100 |
| | indole | | |
| 7-Hydroxy-4,6-dimethyl phthalide | | | 100 |
| 2-Hydroxymethyl-5-hydroxy-γ- | | 501-30-4 | 97 |
| pyrone (kojic acid) | | | |
| Hydrobromide salsolinol | | | 94 |
| 3,4-Dihydroxycinnamic acid | Caffeic | 331-39-5 | 88 |
| | acid | | |
| Physostigmine | | | 87 |

% deglycation: Fluorescence obtained with the active principle versus the fluorescence obtained with the positive control (15 mM aminoguanidine) in %.

### Example 3

### Deglycation of AGEs by Plant Extracts

The preparation of a solution of protein/reducing sugar was carried out according to the method described in Example 1 (paragraph 1).

The compounds from Table 2 were extracts obtained by maceration in water. The compounds were tested at a concentration between 0.01 and 5%, more particularly at 1% by weight of the total composition.

TABLE 2

| | | Part of the | |
|---|---|---|---|
| Common name | Latin name | plant | % deglycation |
| Awati | Maprounea | Leaf | 100 |
| | guyanensis | | |
| Piper mistletoe | Phoradendron | Whole plant | 100 |
| | piperoides | | |
| Jamaica vervain | Stachytarpheta | Leaf | 100 |
| | jamaicensis | | |
| Cecropia | Cecropia obtusa | Leaf | 100 |
| Cecropia | Cecropia obtusa | Bud | 96 |
| Wintergreen | Chimaphila | | 93 |
| | umbellata | | |
| Cherry tree | Prunus cerasus | Bark | 81 |
| Cassia | Senna alata | Leaf | 77 |
| Indian shot | Indian shot | Root | 72 |

% deglycation: Fluorescence obtained with the active principle versus the fluorescence obtained with the positive control (15 mM aminoguanidine) in %.

### Example 4

### Activity of the Substances According to the Present Invention on the Deglycation of Human Collagen

The preparation of a solution of protein/reducing sugar was carried out according to the method described in Example 1 (paragraph 1) with the use of human collagen replacing the BSA.

1—Preparation of Human Collagen.

The extraction of human collagen was carried out from a human biopsy originating from plastic surgery. The collagen obtained in the form of a solution was incubated with a solution of reducing sugar such as glucose, fructose or ribose, and preferably with ribose. The incubation concentrations were the same as described in the preceding examples.

2—Preparation and Test of the Potentially Active Principles

The potentially active principles of the “plant extracts” type and of the “characterized molecules” type were used under the same conditions as those cited in Examples 2 and 3.

3—Results

The molecules that promote the deglycation of AGEs under the conditions described, in a manner at least equivalent to that of the 15 mM aminoguanidine used, were the same as those cited in Examples 2 and 3.

### Examples 5 to 10

### Antiageing Composition that can be Applied Topically

In the following examples “products of the invention” represent the active substances according to the invention and in particular those obtained according to Example 2 or 3.

### Example 5

### Use of the Products of the Invention in Cosmetic or Pharmaceutical Formulations of Oil-in-Water Emulsion Type

Formulation 5a:

| A | Water | qs for 100 |
|---|---|---|
| | Butylene glycol | 2 |
| | Glycerine | 3 |
| | Sodium dihydroxycetyl | 2 |
| | phosphate, | |
| | isopropyl hydroxycetyl ether | |
| B | Glycol stearate | 14 |
| | Triisononaoin | 5 |
| | Octyl cocoate | 6 |
| C | Butylene glycol, | 2 |
| | methylparaben, | |
| | ethylparaben, propylparaben, | |
| | pH adjusted to 5.5 | |
| D | Products of the invention | 0.001-10% |

Formulation 5b:

| A | Water | qs for 100 |
|---|---|---|
| | Butylene glycol | 2 |
| | Glycerine | 3 |
| | Polyacrylamide, isoparaffin, | 2.8 |
| | laureth-7 | |
| B | Butylene glycol, | 2 |
| | methylparaben, | |
| | ethylparaben, propylparaben; | |
| | Phenoxyethanol, | 2 |
| | methylparaben, | |
| | propylparaben, butylparaben, | |
| | ethylparaben | |
| | Butylene glycol | 0.5 |
| D | Products of the invention | 0.001-10% |

Formulation 5c:

| | | |
|---|---|---|
| A | Carbomer | 0.50 |
| | Propylene glycol | 3 |
| | Glycerol | 5 |
| | Water | qs for 100 |
| B | Octyl cocoate | 5 |
| | Bisabolol | 0.30 |
| | Dimethicone | 0.30 |
| C | Sodium hydroxide | 1.60 |
| D | Phenoxyethanol, | 0.50 |
| | methylparaben, | |
| | propylparaben, butylparaben, | |
| | ethylparaben | |
| E | Fragrance | 0.30 |
| F | Products of the invention | 0.001-10% |

### Example 6

### Use of the Products of the Invention in a Formulation of Water-in-Oil Type

| | | |
|---|---|---|
| A | PEG-30 | 3 |
| | dipolyhydroxystearate | |
| | Capric triglycerides | 3 |
| | Cetearyl octanoate | 4 |
| | Dibutyl adipate | 3 |
| | Grape seed oil | 1.5 |
| | Jojoba oil | 1.5 |
| | Phenoxyethanol, | 0.5 |
| | methylparaben, | |
| | propylparaben, butylparaben, | |
| | ethylparaben | |
| B | Glycerine | 3 |
| | Butylene glycol | 3 |
| | Magnesium sulphate | 0.5 |
| | EDTA | 0.05 |
| | Water | qs for 100 |
| C | Cydomethicone | 1 |
| | Dimethicone | 1 |
| D | Fragrance | 0.3 |
| E | Products of the invention | 0.001-10% |

### Example 7

### Use of the Products of the Invention in an Aqueous Gel (Eye Contours, Etc.) Formulation

| A | Water | qs for 100 |
|---|---|---|
| | Carbomer | 0.5 |
| | Butylene glycol | 15 |
| | Phenoxyethanol, methylparaben, | 0.5 |
| | propylparaben, butylparaben, | |
| | ethylparaben | |
| B | Products of the invention | 0.001-10% |

### Example 8

### Use of the Products of the Invention in a Formulation of Shampoo or Shower Gel Type

| | | |
|---|---|---|
| A | Xanthan gum | 0.8 |
| | Water | qs for 100 |
| B | Butylene glycol, | 0.5 |
| | methylparaben, | |
| | ethylparaben, propylparaben | |
| | Phenoxyethanol, | 0.5 |
| | methylparaben, | |
| | propylparaben, butylparaben, | |
| | ethylparaben | |
| C | Citric acid | 0.8 |
| D | Sodium laureth sulphate | 40.0 |
| E | Product of the invention | 0.001-10% |

### Example 9

### Use of the Products of the Invention in a Formulation of a Lipstick Type and Other Anhydrous Products

| | | |
|---|---|---|
| A | Mineral wax | 17.0 |
| | Isostearyl isostearate | 31.5 |
| | Propylene glycol dipelargonate | 2.6 |
| | Propylene glycol isostearate | 1.7 |
| | PEG 8 beeswax | 3.0 |
| | Hydrogenated palm kernel oil | 3.4 |
| | glycerides, | |
| | hydrogenated palm glycerides | |
| | Lanolin oil | 3.4 |
| | Sesame oil | 1.7 |
| | Cetyl lactate | 1.7 |
| | Mineral oil, lanolin alcohol | 3.0 |
| B | Castor oil | qs for 100 |
| | Titanium dioxide | 3.9 |
| | CI 15850:1 | 0.616 |
| | CI 45410:1 | 0.256 |
| | CI 19140:1 | 0.048 |
| | CI 77491 | 2.048 |
| C | Products of the invention | 0.001-5% |

### Example 10

### Use of the Products of the Invention in a Tablet, Ointment or Injectable Formulation

Formulation 10a: Preparation of Tablets

| A | Excipients | In g per tablet |
|---|---|---|
| | Lactose | 0.359 |
| | Saccharose | 0.240 |
| B | Active principle* | 0.001-0.1 |

*The active principle is obtained, for example, according to the extraction process described in the examples, followed by a drying step.

Formulation 10b: Preparation of an Ointment

| A | Excipients | |
|---|---|---|
| | Low-density polyethylene | 5.5 |
| | Liquid paraffin | qs for 100 |
| B | Active principle* | 0.001-0.1 |

*The active principle is obtained, for example, according to the extraction process described in the examples, followed by a drying step.

Formulation 10c: Preparation of an Injectable Formula

| | | |
|---|---|---|
| A | Excipient | |
| | Isotonic saline solution | 5 ml |
| B | Active principle* | 0.001-0.1 g |

*The active principle is obtained, for example, according to the extraction process described in the examples, followed by a drying step.

Phase A and phase B are packaged in separate ampoules and mixed before use.

1-10. (canceled)

11. A method of promoting the deglycation of AGEs in human cutaneous tissue comprising topically applying to the tissue a composition comprising at least one active ingredient which promotes the reversion of the Maillard reaction with respect to AGEs.

12. The method of claim 11, wherein the active ingredient is chosen from the group consisting of 3,5-dimethoxy-4-hydroxycinnamic acid (sinapic acid), trans-3,3′,4′,5,7-pentahydroxyflavane (catechin), oxindole, 3,4-dihydroxyphenylacetic acid (DOPAC), 1,3,5-trihydroxybenzene (phloroglucinol), 4-aminophenol, 2-hydroxybenzoic acid (salicylic acid), 6-hydroxy-2,5,7,8-tetramethylchroman-2-carboxylic acid (trolox), nordihydroguaiaretic acid, 6-methyl-5,7a,13,13b-tetraazapentaphene-8,14-dione, 6-hydroxyindole, salsolinol hydrobromide, 7-hydroxy-4,6-dimethyl phthalide, 3,4-dihydroxycinnamic acid (caffeic acid), physostigmine, 2-hydroxymethyl-5-hydroxy-γ-pyrone (kojic acid), await extract, Jamaica vervain extract, Cecropia extract, wintergreen extract, piper mistletoe extract, cherry tree extract, Cassia extract, Indian shot extract; and mixtures thereof.

13. The method of claim 11 wherein the composition is a cosmetic composition.

14. The method of claim 12 wherein said active ingredient is a await extract, Jamaica vervain extract, Cecropia extract, wintergreen extract, piper mistletoe extract, cherry tree extract, Cassia extract, or Indian shot extract; and said active ingredient comprises between 0.001 and 10% by weight of the total composition.

15. The method of claim 12 wherein the active ingredient is 3,5-dimethoxy-4-hydroxycinnamic acid (sinapic acid), trans-3,3′,4′,5,7-pentahydroxyflavane (catechin), oxindole, 3,4-dihydroxyphenylacetic acid (DOPAC), 1,3,5-trihydroxybenzene (phloroglucinol), 4-aminophenol, 2-hydroxybenzoic acid (salicylic acid), 6-hydroxy-2,5,7,8-tetramethylchroman-2-carboxylic acid (trolox), nordihydroguaiaretic acid, 6-methyl-5,7a,13,13b-tetraazapentaphene-8,14-dione, 6-hydroxyindole, salsolinol hydrobromide, 7-hydroxy-4,6-dimethyl phthalide, 3,4-dihydroxycinnamic acid (caffeic acid), physostigmine, 2-hydroxymethyl-5-hydroxy-γ-pyrone (kojic acid) and said active ingredient comprises between 10⁻⁷ and 1% by weight of the total composition.

16. A method of screening active principles that promote the deglycation of AGEs, comprising:
a) the production of AGEs;
b) bringing the AGEs into contact with a substance to be screened for its activity with respect to the deglycation of AGEs; and
c) the selection of at least one active principle that promotes the deglycation of AGEs.

17. A screening method according to claim 16, wherein that step a) comprises an incubation of at least one type of protein from the skin or from blood vessel walls, for example human collagen, in the presence of a sugar under conditions that enable the formation of AGEs.

18. The screening method of claim 16 wherein step b) comprises the incubation of AGEs in the presence of at least one substance to be screened at a temperature between 40 and 60° C.

19. A cosmetic composition, that can be applied topically or as a food supplement, comprising an active ingredient that promotes the reversion of the Maillard reaction with respect to AGEs in the skin selected from the group consisting of oxindole, 3,4-dihydroxyphenylacetic acid (DOPAC), 1,3,5-trihydroxybenzene (phloroglucinol), 4-aminophenol, 2-hydroxybenzoic acid (salicylic acid), 6-hydroxy-2,5,7,8-tetramethylchroman-2-carboxylic acid (trolox), nordihydroguaiaretic acid, 6-methyl-5,7a,13,13b-tetraazapentaphene-8,14-dione, 6-hydroxyindole, salsolinol hydrobromide, 7-hydroxy-4,6-dimethyl phthalide, physostigmine, await extract, Jamaica vervain extract, Cecropia extract, wintergreen extract, piper mistletoe extract, cherry tree extract, Cassia extract, Indian shot extract and any mixture thereof.

20. The cosmetic composition of claim 19 further comprising a compound that inhibits glycation, or that promotes the protection of skin proteins, or enzyme inhibitors, or substances that reduce the amount of sugar available to participate in the glycation reaction.

21. A pharmaceutical composition comprising an active ingredient that promotes the reversion of the Maillard reaction with respect to AGEs in skin selected from the group consisting of oxindole, 3,4-dihydroxyphenylacetic acid (DOPAC), 1,3,5-trihydroxybenzene (phloroglucinol), 4-aminophenol, 2-hydroxybenzoic acid (salicylic acid), 6-hydroxy-2,5,7,8-tetramethylchroman-2-carboxylic acid (trolox), nordihydroguaiaretic acid, 6-methyl-5,7a,13,13b-tetraazapentaphene-8,14-dione, 6-hydroxyindole, salsolinol hydrobromide, 7-hydroxy-4,6-dimethyl phthalide, physostigmine, await extract, Jamaica vervain extract, Cecropia extract, wintergreen extract, piper mistletoe extract, cherry tree extract, Cassia extract, Indian shot extract and mixtures thereof.

22. The cosmetic composition of claim 21 further comprising a compound that inhibits glycation, or that promotes the protection of skin proteins, or enzyme inhibitors, or substances that reduce the amount of sugar available to participate in the glycation reaction.
